(19) United States
(12) Patent Application Publication
(10) Pub. No.: US 2013/0260416 A1
(43) Pub. Date: Oct. 3, 2013

(54) METHOD AND SYSTEM FOR DETECTING MICROCYSTIN FROM REFLECTED LIGHT

(71) Applicant: BOWLING BREEN STATE UNIVERSITY, Bowling Green, OH (US)

(72) Inventor: Robert K. Vincent, Bowling Green, OH (US)

(21) Appl. No.: 13/853,975

(22) Filed: Mar. 29, 2013

Related U.S. Application Data
(60) Provisional application No. 61/618,142, filed on Mar. 30, 2012.

(51) Int. Cl. C12Q 1/04

(52) U.S. Cl. CPC C12Q 1/04
USPC 435/34; 435/288.7; 702/19

(57) ABSTRACT

The present invention relates to a method of detecting microcystin in water from reflected light, and also includes devices for the measurement, calculation and transmission of data relating to that method.

### CROSS-REFERENCE TO RELATED APPLICATIONS

This application is a non-provisional of and, pursuant to 35 U.S.C. §119(e), claims the benefit of, U.S. Provisional Patent Application Ser. No. 61/618,142, filed on Mar. 30, 2012 under 35 U.S.C. §112(b), which is pending as of the filing date of this application. Application Ser. No. 61/618,142 is hereby incorporated by reference in its entirety to the extent permitted by law.

### STATEMENT REGARDING GOVERNMENTAL INTEREST

The present invention was made through funding from the National Oceanic and Atmospheric Administration (NOAA), grant number NA09OAR4170223. The United States Government has certain rights in the invention.

### BACKGROUND OF THE INVENTION

The present invention relates to a method of detecting microcystin content in water from reflected light.

In many instances it is desirable to be able to detect the presence of toxins produced by microorganisms in water, particularly bodies of water that serve as a source for drinking water or that may serve as a site for recreation, such as for swimming, boating, water sports, and fishing. Many of these toxins in high concentrations can be harmful to the public and to the environment generally.

It is particularly desirable to be able to be able to detect the presence toxins produced by microorganisms in water in a manner that is convenient and provides relatively immediate results so that the public may be warned or other actions taken to avoid or eliminate contamination of the assayed water.

One such toxin is microcystin, a peptide hepatotoxin produced by Microcystis spp. The toxin is harmful to waterfowl or other animals that might drink the untreated water. Microcytsin has also been identified as a tumor promoter, making long term ingestion of even low levels of the toxin of concern. The World Health Organization (WHO) has set an international sporting lake advisory standard for microcystin toxin of 20 μg/L, one of a very few internationally accepted advisory limits concerning water quality.

With the availability of LANDSAT 7 imagery for every 16-day overpass period, one embodiment of the invention is intended to develop a LANDSAT TM algorithm for detecting various levels of microcystin in surface waters. With the availability of LANDSAT Thematic Mapper™ imagery featuring an overpass cycle of every 16 days (8-day intervals, if both LANDSAT 5 and 7 are employed), a goal of one embodiment of the present invention is to develop a set of algorithms, methods of their use, and devices for detecting microcystin in surface waters, based on a unique spectral signature produced by microsytin.

The present invention allows for a lake manager or Ohio EPA manager to hire the service of a company to apply the algorithm of the invention instead of having to collect one or two water grab samples after a cyanobacteria bloom is suspected in a lake, as is done now by Ohio EPA, for example. In one embodiment of the present invention, only LANDSAT TM data input into the algorithm of the present invention is required to determine which parts of a lake are below the WHO sporting lake advisory limit or above it, which would require ordering people off the lake. The information could be available for far less expense and be available within 24 hours and have 5 measurements per acre over an entire lake made by an algorithm in accordance with the present invention.

In addition to the features mentioned above, objects and advantages of the present invention will be readily apparent upon a reading of the following description and through practice of the present invention.

### SUMMARY OF THE INVENTION

In general terms, the present invention includes a method of determining the presence of microcystin in water as well as a measurement method followed by transmission of data to a remote processing site.

The invention includes a method of determining the presence of microcystin in water from light reflected therefrom. The method comprises the steps of: (a) obtaining a measurement of reflected light from the water, the measurement comprising a measurement of the respective amount of light in at least four frequency ranges; and (b) relating the approximate amount of microcystin in the water to the respective amounts of light by applying an algorithm relating the respective amounts of light in the at least four frequency ranges to the amount of microcystin in the water.

In a preferred embodiment, the measurement of reflected light from the water, the measurement comprising a measurement of the respective amount of light in at least four frequency ranges (i) from about 0.45 μm to about 0.52 μm; (ii) from about 0.63 μm to about 0.69 μm; (iii) from about 0.76 μm to about 0.90 μm; and (iv) from about 1.55 μm to about 1.75 μm; and (b) relating the approximate amount of microcystin in the water to the respective amounts of light by applying an algorithm relating the respective amounts of light in the at least four frequency ranges to the amount of microcystin in the water.

Preferably, the measurement of the amount of light in the at least four frequency ranges comprises the measurement, respectively, of: (i) LANDSAT Thematic Mapper (“TM”) band 1, (ii) LANDSAT TM band 3, (iii) LANDSAT TM band 4, and (iv) LANDSAT TM band 5, and wherein the algorithm is any algorithm selected from the group consisting of: X≈K₁+K₂×(R31)+K₃×(R43)+K₄×(R51), wherein X is the approximate amount of microcystin expressed micrograms (μg) per liter (L) and wherein:
K₁ is a value in the range of from about −1 to about −89.7;
K₂ is a value in the range of from about 1 to about 186.99;
K₃ is a value in the range of from about 1 to about 132.99;
K₄ is a value in the range of from about −1 to about −179;
R31 is the value of LANDSAT TM band 3 divided by LANDSAT TM band 1, after subtraction for atmospheric haze separately in each band;
R43 is the value of LANDSAT TM band 4 divided by LANDSAT TM band 3, after subtraction for atmospheric haze separately in each band; and
R51 is the value of LANDSAT TM band 5 divided by LANDSAT TM band 1, after subtraction for atmospheric haze separately in each band.

More preferably wherein X is the approximate amount of microcystin expressed micrograms (μg) per liter (L) and wherein:
K₁ is a value in the range of from about −25 to about −75;
K₂ is a value in the range of from about 50 to about 150;
K₃ is a value in the range of from about 45 to about 100;
K₄ is a value in the range of from about −50 to about −150;
R31 is the value of LANDSAT TM band 3 divided by LANDSAT TM band 1, after subtraction for atmospheric haze separately in each band;
R43 is the value of LANDSAT TM band 4 divided by LANDSAT TM band 3, after subtraction for atmospheric haze separately in each band; and
R51 is the value of LANDSAT TM band 5 divided by LANDSAT TM band 1, after subtraction for atmospheric haze separately in each band.

Still more preferably wherein X is the approximate amount of microcystin expressed micrograms (μg) per liter (L) and wherein:
K₁ is a value in the range of from about −50 to about −70;
K₂ is a value in the range of from about 85 to about 115;
K₃ is a value in the range of from about 55 to about 80;
K₄ is a value in the range of from about −75 to about −100;
R31 is the value of LANDSAT TM band 3 divided by LANDSAT TM band 1, after subtraction for atmospheric haze separately in each band;
R43 is the value of LANDSAT TM band 4 divided by LANDSAT TM band 3, after subtraction for atmospheric haze separately in each band; and
R51 is the value of LANDSAT TM band 5 divided by LANDSAT TM band 1, after subtraction for atmospheric haze separately in each band.

Still more preferably wherein X is the approximate amount of microcystin expressed micrograms (μg) per liter (L) and wherein:
K₁ is a value of about −63.2388;
K₂ is a value of about 96.118;
K₃ is a value of about 68.362;
K₄ is a value of about −92.006;
R31 is the value of LANDSAT TM band 3 divided by LANDSAT TM band 1, after subtraction for atmospheric haze separately in each band;
R43 is the value of LANDSAT TM band 4 divided by LANDSAT TM band 3, after subtraction for atmospheric haze separately in each band; and
R51 is the value of LANDSAT TM band 5 divided by LANDSAT TM band 1, after subtraction for atmospheric haze separately in each band.

The method according to the present invention is such that the calculated value of microcystin (X) correlates to the actual measured amount of the microcystin in the water by a correlation value in excess of 60% and as high as in excess of 80%.

The present invention may additionally comprise the step of generating a report of the approximate amount of microcystin. This may be done using electronics adapted to digitize and process the data using an appropriate algorithm, such as that described herein. For instance, the report may include an estimate the amount of microcystin in micrograms (μg) per liter (L) in the water.

The method of the present invention may also include the step of transmitting data relating to the approximate amount of microcystin micrograms (μg) per liter (L) in the water to a site remote from the site where the measurement takes place. This may be done using any transmission method including land line or wireless transmission. This is also used advantageously where the reflected light is sensed remotely by aircraft, satellite, boat or buoy. Processing of the data may take place at the site of light uptake or may be carried out at a remote location after transmission of the raw data. The estimated microcystin report may be sent to public authorities, such as police departments, fire and rescue departments or life guard services to warn swimmers, boaters, sportsman or the public at large that a given body of water, or portion thereof, likely contains elevated/dangerous levels of microcystin.

The invention also includes an apparatus for determining the presence of microcystin in water from light reflected therefrom, the device comprising: (a) a measurement device adapted to measure reflected light from the water, the measurement comprising a measurement of the respective amount of light in at least four frequency ranges (i) from about 0.45 μm to about 0.52 μm; (ii) from about 0.63 μm to about 0.69 μm; (iii) from about 0.76 μm to about 0.90 μm; and (iv) from about 1.55 μm to about 1.75 μm; and (b) a processor capable of relating the approximate amount of microcystin in the water to the respective amounts of light by applying an algorithm relating the respective amounts of light in the at least four frequency ranges to the amount of microcystin in the water.

It is preferred that the at least four frequency ranges comprise, respectively: (i) LANDSAT TM band 1, (ii) LANDSAT TM band 3 and (iii) LANDSAT TM band 4, and (iv) LANDSAT TM band 5, and wherein the algorithm is any algorithm selected from the group consisting of: X≈K₁+K₂×(R31)+K₃×(R43)+K₄×(R51), wherein X is the approximate amount of microcystin expressed in micrograms (μg) per liter (L) and wherein:
K₁ is a value in the range of from about −1 to about −89.7;
K₂ is a value in the range of from about 1 to about 186.99;
K₃ is a value in the range of from about 1 to about 132.99;
K₄ is a value in the range of from about −1 to about −179;
R31 is the value of LANDSAT TM band 3 divided by LANDSAT TM band 1, after subtraction for atmospheric haze separately in each band;
R43 is the value of LANDSAT TM band 4 divided by LANDSAT TM band 3, after subtraction for atmospheric haze separately in each band; and
R51 is the value of LANDSAT TM band 5 divided by LANDSAT TM band 1, after subtraction for atmospheric haze separately in each band.

More preferably wherein X is the approximate amount of microcystin expressed micrograms (μg) per liter (L) and wherein:
K₁ is a value in the range of from about −25 to about −75;
K₂ is a value in the range of from about 50 to about 150;
K₃ is a value in the range of from about 45 to about 100;
K₄ is a value in the range of from about −50 to about −150;
R31 is the value of LANDSAT TM band 3 divided by LANDSAT TM band 1, after subtraction for atmospheric haze separately in each band;
R43 is the value of LANDSAT TM band 4 divided by LANDSAT TM band 3, after subtraction for atmospheric haze separately in each band; and
R51 is the value of LANDSAT TM band 5 divided by LANDSAT TM band 1, after subtraction for atmospheric haze separately in each band.

Still more preferably wherein X is the approximate amount of microcystin expressed micrograms (μg) per liter (L) and wherein:
K₁ is a value in the range of from about −50 to about −70;
K₂ is a value in the range of from about 85 to about 115;
K₃ is a value in the range of from about 55 to about 80;
K₄ is a value in the range of from about −75 to about −100;
R31 is the value of LANDSAT TM band 3 divided by LANDSAT TM band 1, after subtraction for atmospheric haze separately in each band;
R43 is the value of LANDSAT TM band 4 divided by LANDSAT TM band 3, after subtraction for atmospheric haze separately in each band; and
R51 is the value of LANDSAT TM band 5 divided by LANDSAT TM band 1, after subtraction for atmospheric haze separately in each band.

Still more preferably wherein X is the approximate amount of microcystin expressed micrograms (μg) per liter (L) and wherein:
K₁ is a value of about −63.2388;
K₂ is a value of about 96.118;
K₃ is a value of about 68.362;
K₄ is a value of about −92.006;
R31 is the value of LANDSAT TM band 3 divided by LANDSAT TM band 1, after subtraction for atmospheric haze separately in each band;
R43 is the value of LANDSAT TM band 4 divided by LANDSAT TM band 3, after subtraction for atmospheric haze separately in each band; and
R51 is the value of LANDSAT TM band 5 divided by LANDSAT TM band 1, after subtraction for atmospheric haze separately in each band.

It is preferred that the apparatus is capable of performing such that the calculated value of microcystin correlates to the actual measured amount of the microcystin in the water by a correlation value in excess of 60, and most preferably by a correlation value in excess of 80.

The apparatus may additionally include a report generator adapted to generate a report of the approximate amount of microcystin in the water. Such a report generator may be any device that is adapted to place the data into a tangible medium, such as a printer, CD burner, flash memory, magnetic storage media, etc.

The apparatus may be mounted anywhere reflected light may be gathered, such as, but not limited to, on a ship, a buoy, a shore tower, an airplane, weather balloon, or drone.

The apparatus may be made to a size that can be conveniently handheld using known miniaturization techniques and materials.

The apparatus may additionally include a transmitter adapted to transmit data relating to the approximate amount of microcystin in the water from the processor to a site remote from the site where the measurement takes place. Such a transmitter may include those adapted to send data such as through land line or wireless transmission, including telephone, internet, cell phone, radio and the like.

The measurement device may be any device adapted to sense and record and/or transmit the light frequencies described above. Examples include photosensors, cameras, digital cameras and video cameras, etc.

The processor may be any data processing device having programming instructions for applying the algorithm, such as preferably a microprocessor.

It is preferred that the algorithm comprises a linear relationship between the approximate amount of microcystin in the water and sum of (a) the ratio of a first frequency to a second frequency, (b) the ratio of a third frequency to the first frequency and (c) the ratio of a fourth frequency to the second frequency.

The measurement device may be a satellite or be any appropriate device placed in any position from which it can sense the required light frequencies, such as on a buoy, a boat, a light house or similar dedicated tower structure, such as an elevated lifeguard house. The measurement device may also be in the form of a handheld device, such as a camera connected to a processor for processing the recorded light frequencies, the device may also be in the form of a device similar to a personal digital assistant with light recording and processing functions.

Another variation of the invention is a system using transmission of light measurement data to processor at a different location, recognizing that the processing may be done at a different location than the light sensing/recording.

In general terms, this variation is a system for determining the presence of microcystin in water from light reflected therefrom, the device comprising (a) a measurement device adapted to measure reflected light from the water, the measurement comprising a measurement of the respective amount of light in at least four frequency ranges (i) from about 0.45 μm to about 0.52 μm; (ii) from about 0.63 μm to about 0.69 μm; (iii) from about 0.76 μm to about 0.90 μm; and (iv) from about 1.55 μm to about 1.75 μm and (b) a processor at the remote site and capable of relating the amount of microcystin in the water to the respective amounts of light by applying an algorithm relating the respective amounts of light in the at least three frequency ranges to the amount of microcystin in the water.

The invention also includes a method of developing an apparatus for determining the presence of microcystin in water from light reflected therefrom, the device comprising (a) obtaining a measurement of reflected light from the water, the measurement comprising a measurement of the respective amount of light of at least two frequencies; (b) developing an algorithm relating the respective amounts of light in the at least two frequencies to the amount of microcystin in the water through linear regression analysis; (c) producing a processor capable of relating the approximate amount of microcystin in the water to the respective amounts of light by applying an algorithm relating the respective amounts of light in the at least two frequency ranges to the amount of microcystin in the water; and (d) providing a measurement device adapted to measure reflected light from the water and adapted to provide data relating to the measurement to the processor.

It is also preferred that the at least two frequencies comprises a LANDSAT TM band, and most preferably comprises a measurement of LANDSAT TM band 1, LANDSAT TM band 3, LANDSAT TM band 4 and LANDSAT TM band 5. Also preferably, the algorithm includes a ratio of at least two frequencies.

Reflected light for any of the embodiments described herein includes light reflected from any non-monochromatic source of light, such as, but not limited to sunlight.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Novel features and advantages of the present invention, in addition to those mentioned above, will become apparent to those skilled in the art from a reading of the following detailed description in conjunction with the accompanying drawings summarized as follows:

FIG. 1 is a graphical plot of the toxin microcystin measured in water samples collected close to the time of LANDSAT TM overpass over the Western basin of lake Erie on Sep. 25, 2008 plotted against results using an algorithm in accordance with one embodiment of the present invention.

FIG. 2 is a photograph illustrating the LANDSAT 7 TM natural color (right) and total microcystin (left) images (redder being greater) of the Western basin of Lake Erie (form LANDSAT TM data) in accordance with one embodiment of the present invention.

FIG. 3 is a photograph illustrating the LANDSAT 7 TM natural color (right) and total microcystin (left) images (redder being greater) in the Maumee Bay area (Toledo and Maumee, Ohio) in accordance with one embodiment of the present invention.

FIG. 4 is a photograph illustrating microcystin mapping (red means more) from LANDSAT TM in higher zoom of Maumee Bay area in accordance with one embodiment of the present invention.

### DETAIL DESCRIPTION OF PREFERRED EMBODIMENT(S)

The preferred system herein described is not intended to be exhaustive or to limit the invention to the precise forms disclosed. They are chosen and described to explain the principles of the invention and the application of the method to practical uses so that others skilled in the art may practice the invention.

The present invention includes a system using an algorithm for converting LANDSAT TM multispectral signals into images showing different values of amounts of microcystin in water. This system and method were tested in Lake Erie and its wider tributaries to detect microcystin in the waters of Lake Erie in order to analyze the changes in water populations as they affect human activities. By gathering water samples during the period of time the satellite passes over Lake Erie and applying test kits, the level of microcystin was determined.

The method of the present invention may be carried out using any sensing appropriate light sensing devices adapted to capture the algorithm-relevant frequencies as described herein, including satellite and surface sensors for detection of microcystin.

An algorithm that may be used in the present invention, which may be carried out by computer instructions for producing a particular type of image that can be used to map a particular substance from a remote sensing platform in space, in an aircraft, or on the ground, may be determined as follows.

LANDSAT Thematic Mapper (TM) is a sensor that has 8 spectral bands, 6 of which have a 30-meter spatial resolution and which detect visible and infrared radiation (sunlight) reflected off the Earth's surface. The following bands were employed, with the wavelength limits (in micrometers, or μm) of their spectral band-widths given below for the LANDSAT 7 version of TM, called ETM+, and the LANDSAT 4 and 5 versions called TM:

TABLE 1
TM and ETM+ Spectral Bandwidths
Bandwidth (μ) Full Width - Half Maximum

| | Band 1 | Band 2 | Band 3 | Band 4 | Band 5 | Band 6 | Band 7 | Band 8 |
|---|---|---|---|---|---|---|---|---|
| | Plot | Plot | Plot | Plot | Plot | Plot | Plot | Plot |
| Sensor | Data | Data | Data | Data | Data | Data | Data | Data |
| TM | 0.45-0.52 | 0.52-0.60 | 0.63-0.69 | 0.76-0.90 | 1.55-1.75 | 10.4-12.5 | 2.08-2.35 | N/A |
| ETM+ | 0.45-0.52 | 0.53-0.61 | 0.63-0.69 | 0.78-0.90 | 1.55-1.75 | 10.4-12.5 | 2.09-2.35 | .52-.90 |

For instance, band 2 of the LANDSAT 7 version of the TM sensor (called ETM+) has wavelength limits of 0.53-0.61 μm, band 3 has limits of 0.63-0.69 μm, and band 4 has limits of 0.78-0.90 μm. When mapping microcystin in Lake Erie and its tributaries with LANDSAT 7 data, it had to be determined which or how many of bands 1-5 and 7 (which have 30-m spatial resolution and relatively narrow spectral bands, as opposed to the 60-m spatial resolution of band 6 and the relatively wide band-width of the 15-m-resolution band 8) to use. A mathematical procedure (multiple regressions) was applied to seek the best combinations of those bands to correlate with microcystin. It was determined that the use of the single band radiances (even if they were reduced to spectral reflectances from theoretical atmospheric models) as inputs to this procedure, the resulting algorithm would not be very robust (i.e., repeatable under different solar illumination and atmospheric conditions). Therefore, spectral ratios (ratios of spectral bands, after empirical correction for atmospheric haze through a process referred to as “dark object subtraction” were input to the mathematical procedure for each pixel from which a water sample had been collected. These 15 non-reciprocal ratios (R21, R31, R32, R41, . . . R75) became the dependent variables and microcystin became the independent variable, which was the result of lab analysis of the water samples. For the LANDSAT 7 overpass, 30 water samples were collected, which were measured for both phycocyanin and microcystin content. The best subsets of spectral ratios were determined, and then the ones with the highest R² (Adjusted) values were tested to see if they passed the Durbin-Watson test. The model with the highest R² (Adjusted) that also passed the Durbin-Watson test was the model that was considered to be the best.

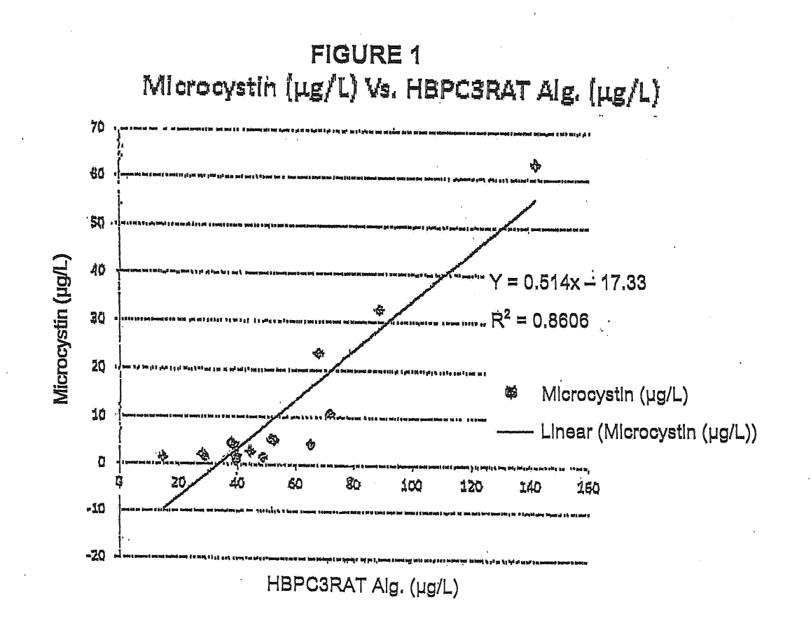

A microcystin algorithm has been invented that uses the HBPC3RAT algorithm (invented earlier, see, e.g. U.S. Pat. No. 8,367,369 to Vincent; the 3RAT indicates that it employs 3 dark-object-subtracted spectral ratios of LANDSAT TM spectral bands) to calculate microcystin content (MC) in a manner that is more accurate for the higher amounts of microcystin (the LR variety of microcystin), because the HBPC3RAT algorithm was designed for mapping phycocyanin content (PC) of high blooms. It has problems with absolute accuracy in very low ranges of PC, but in the following two cases, two LANDSAT TM overflight data sets from 2009 and 2010, it worked well because in most cases it at least recognizes those values as being below the WHO sporting lake advisory limit of 20 μg/L of MC. FIG. 1 shows a graphical plot of the toxin microcystin measured in water samples collected close to the time of LANDSAT TM overpass over the Western Basin of Lake Erie on Sep. 25, 2008, versus the new MC algorithm results. The new MC algorithm is explained by the following equation (s):

High Bloom Microcystin Content (HBMC) in μg/L=X;

X=0.514×(HBPC3RAT)−17.133, where HBPC3RAT=−89.7+187(R31)+133(R43)−179(R51), or

X=−63.2388+96.118(R31)+68.362(R43)−92.006(R51),

and Rij is the dark-object-subtracted (atmospheric haze and sensor additive offset corrected) spectral ratio of the ith and jth spectral bands, in this case of the LANDSAT TM satellite sensor.

Dark-object-subtracted spectral ratios are described in the text book Fundamentals of Geological and Environmental Remote Sensing, Prentice Hall, Upper Saddle River, N.J., 400 pp., 1997, by Dr. Robert K. Vincent, Dept. of Geology, Bowling Green State University, Bowling Green, Ohio, which is hereby incorporated by reference in its entirety to the extent permitted by law.

The algorithms of the preferred embodiments are not very absolutely accurate at low values of microcystin content (MC), i.e. below 10 μg/L, but the curve has an R² value of 0.86 and would have correctly recognized all three of the water samples that had MC greater than or equal to 20 μg/L, but would have recognized one with 11 μg/L as having about 20 μg/L. It would have also correctly recognized that the other 14 water samples were below the WHO sporting lake advisory limit of 20 μg/L. When the algorithms are applied to the water samples collected near the time of a LANDSAT TM overpass on Sep. 4, 2009 for which microcystin data were measured, all 30 water samples were measured in the lab from the water samples to be well below 20 μg/L, and they were all below that level according to the microcystin algorithm from the HBMC equation described above. The same is true of the 30 water samples from Sep. 15, 2010; all measured samples were well below 20 μg/L, as were the microcystin algorithm results from the HBMC equation above. Thus, the microcystin algorithm from the HBMC equation correctly classified 76 of 77 water sampling sites as having microcystin values exceeding 20 μg/L or not, with one false alarm for a site where MC was measured to be 11 μg/L, and the microcystin algorithm measured it as 20 μg/L.

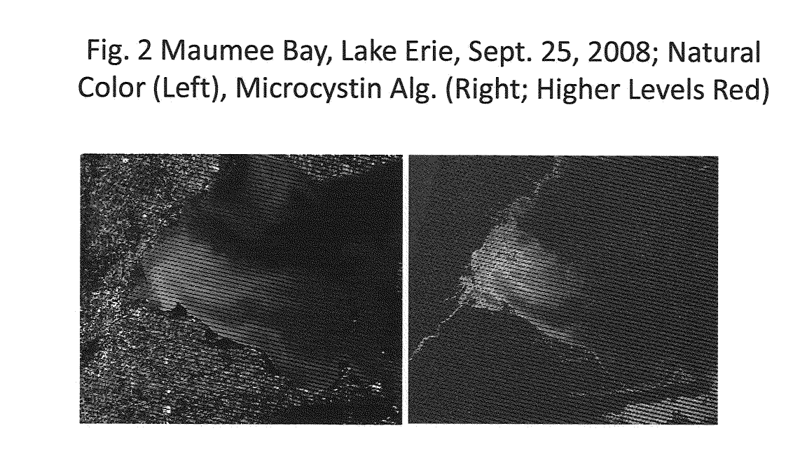

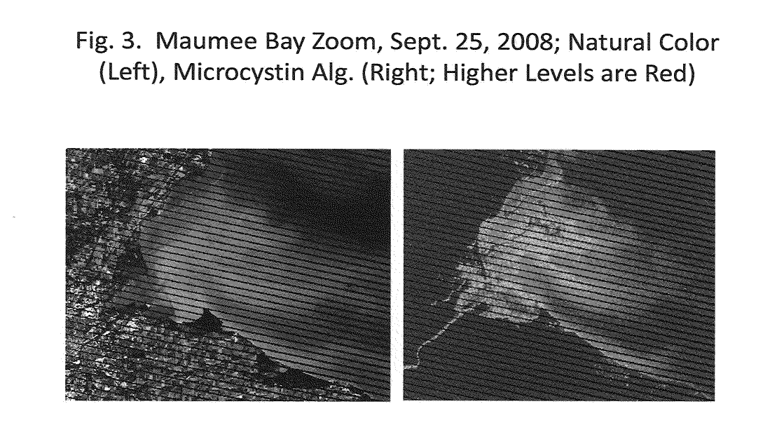

When the HBMC equation was applied to the Sep. 25, 2008 LANDSAT 7 TM frame, FIG. 2 resulted. The color key for the data shown in FIGS. 2 and 3 is as follows:

| | Microcystin content in water |
|---|---|
| Color | (μg/L) |
| Red | 90-150 |
| Orange | 80-89 |
| Yellow | 60-79 |
| Blue-Green | 45-59 |
| Light Blue | 1-44 |
| Dark Blue | ≦0 |

FIG. 1 is a graphical plot of the toxin microcystin measured in water samples collected close to the time of LANDSAT TM overpass over the Western basin of Lake Erie on Sep. 25, 2008 plotted against results using an algorithm in accordance with one embodiment of the present invention. FIG. 1 acts as a reminder that only at the high levels (above about 20 μg/L) are the estimates expected to be reasonably accurate in an absolute sense. The methods in accordance with the embodiments of the present invention allow for a way of segregating ≧20 μg/L areas from ≦20 μg/L areas, because 20 μg/L is the WHO advisory limit for sporting lakes.

FIG. 2 is a photograph illustrating the LANDSAT 7 TM natural color (right) and total microcystin (left) images (redder being greater) of the Western basin of Lake Erie (form LANDSAT TM data) in accordance with one embodiment of the present invention.

FIG. 3 is a photograph illustrating the LANDSAT 7 TM natural color (right) and total microcystin (left) images (redder being greater) in the Maumee Bay area (Toledo and Maumee, Ohio) in accordance with one embodiment of the present invention. The red trendrils are windrows of cyanobacterial algae bloom, which suggests that the wind and currents may have lysed (crushed the cells) of the cyanobacteria sufficiently to release both phycocyanin (the pigment) and microcystin (the toxin).

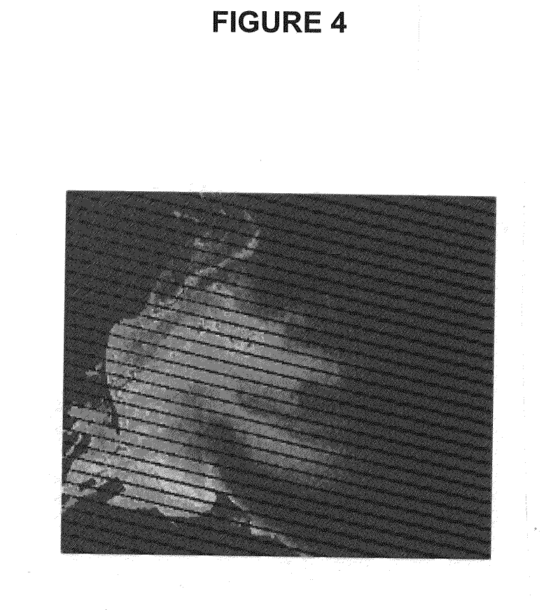

FIG. 4 is a photograph illustrating microcystin mapping (red means more) from LANDSAT TM in higher zoom of Maumee Bay area in accordance with one embodiment of the present invention. Dark blue shows at ≦20 μg/L and everything above approximately 60 μg/L is shown as red. Every part of Western Lake Erie that is not dark blue should have been placed off-limits for water sports, according to the WHO sporting lake advisory.

Also found was that for LANDSAT TM data sets collected in 2011, there were 99 water samples collected for which we had a water reflectance from satellite (not under a cloud or under a null pixel from scan-line-loss corrections by USGS), and the Microcystin Content algorithm, which we are now calling the High Bloom Microcystin Content (HBMC) algorithm, was correct in saying that all 99 of those pixels were below the 20 ppb of Microcystin Content (the WHO advisory limit for warning people off the lake for boating). The closest value to that was about 9 ppb. We did find a few small places outside of Lake Erie in that same (Aug. 17, 2011) showed more than 20 ppc of MC, but they were in about 5 small lakes that were 2.5 acres in size or less, and all had some “blue-green-looking” fringes near their shores in a Google Earth aerial photo that was collected in May, 2012, about 9 months after the Aug. 17, 2011 overpass. This was the only algorithm that did well on these tests of the 2011 data sets, which we showed evidence for being incorrectly calibrated by USGS, and it is because this MC algorithm is only concerned with whether values are greater than or equal to 20 ppb.

The HBMC algorithm could become the most practical and important algorithm developed for mapping water quality by remote sensing methods int eh world because the WHO sporting lake limit with the toxin microcystin is one of the few internationally recognized standards for water quality.

### REFERENCES

Additional background for the invention is provided by the following references which are hereby incorporated by reference to the extent permitted by law.
U.S. Pat. No. 8,367,369 to Vincent.
U.S. Pat. No. 7,132,254 to Vincent.
U.S. Pat. No. 8,417,460 to Vincent.
Abbott, M. R., Chelton, D. B. (1991). Advances in passive remote sensing of the ocean. Geophysics Supplement: 571-589.
Brittain, S. M., Wang, J., Babcock-Jackson, L., Carmichael, W. W., Rinehart, K. L., Culver, D. A., (2000). Isolation and characterization of microcystins, cyclic heptapeptide hepatotoxins from a Lake Erie strain of Microcystis aeruginosa. Journal of Great Lakes Research, 26, 241-249.
Brunberg, A.-K., Blomqvist, P. (2003). Recruitment of Microcystis (Cyanophyceae) from lake sediments: The importance of littoral inocula. Journal of Phycology, 39, 58-63. [0163] Budd, J. W., Beeton, A. M., Stumpf, R. P., Culver, D. A., Kerfoot, W. C. (2002). Satellite observations of Microcystis blooms in western Lake Erie. Verhandlungen Internationale Vereinigung fur theoretische and angewandte Limnologie, 27, 3787-3793.
Carmichael, W. W. 2001. Health effects of toxin-producing cyanobacteria: “The CyanoHABs”. Human and Ecological. Risk Assessment, 7, 1393-1407.
Cowles, T. J., Desiderio, R. A. and Neuer S. (1993). In situ characterization of phytoplankton from vertical profiles of fluorescence emission spectra. Marine Biology, 115, 217-222.
Deane, J. A., Strachan, I. M., Saunders, G. W., Hill, D. R. A., McFadden, G. I. (2002). Cryptomonad evolution: nuclear 18S rDNA phylogeny versus cell morphology and pigmentation. Journal of Phycology, 38, 1236-1244.
Dekker, A. G. (1993). Detection of optical water quality parameters for eutrophic waters by high resolution remote sensing. Ph.D Thesis, Free University, Amsterdam.
Durbin, J., and Watson, G. S. (1951). Testing for serial correlation in least squares regression. II. Biometrica, 38, 159-178.
Falconer, I. R., Humpage, A. R. (1996). Tumour production by cyanobacterial toxins. Phycologia, 35 (6 Suppl), 74-79.
Galat, D. L, Verdin, J. P. (1989). Patchiness, collapse and succession of a cyanobacterial bloom evaluated by synoptic sampling and remote sensing. Journal of Plankton Research, 11, 925-948.
Galat, D. L., Verdin, J. P., Sims, L. L. (1990). Large-scale patterns of Nodularia spumigena blooms in Pyramid Lake, Nev., determined from Landsat imagery: 1972-1986. Hydrobiologia, 197, 147-164.
Gitelson, A., Garbuzov, G., Szilagyi, F., Mittenzwey, K. H., Karnieli, A., Kaiser, A. (1993). Quantitative remote sensing methods for real-time monitoring of inland waters quality. International Journal of Remote Sensing, 14, 1269-1295.
Green, S., 2003, http://www.ucd.ie/.about.app-phys/stuart/MODEL.HTM, The Effect of Chlorophyll Concentration on Airborne Hyperspectral Reflectance.
Hill, D. R. A., Rowan, K. S. (1989). The biliproteins of the Cryptophyceae. Phycologia, 28, 455-463.
Lake Erie LaMP (2002). The Lake Erie Lakewide Management Plan. J. Letterhos and J. Vincent (Eds.), Environment Canada, Ontario Region and U.S. Environmental Protection Agency, Region 5.
MacColl, R., Guard-Friae, D. (1987). Phycobiliproteins. Boca Raton, Fla.: CRC Press
Makarewicz, J. C. (1993). Phytoplankton biomass and species composition in Lake Erie, 1970 to 1987. Journal of Great Lakes Research, 19, 258-274.
Millie, D. F., Baker, M. C., Tucker, C. S., Vinyard, B. T., Dionigi, C. P. (1992). High-resolution airborne remote sensing of bloom-forming phytoplankton. Journal of Phycology, 28, 281-290.
Richardson, L. L, Bachoon, D., Ingram-Willey, V., Ghee, Chow C., Weinstock, K. (1991). Remote sensing of the biological dynamics of large-scale salt evaporation ponds. Pages 611-623 in Proceedings of the International Symposium on Remote Sensing of Environment; 27-31 May; Rio de Janeiro, Brazi.
Richardson, L. L. (1996). Remote sensing of algal bloom dynamics. BioScience, 44, 492-501.
Rowan, K. S. (1989). Photosynthetic pigments of algae. Cambridge University Press.
Smith, R. C., Baker, K. S. (1982). Oceanic chlorophyll concentrations as determined by satellite (Nimbus-7 Coastal Zone Color Scanner). Marine Biology, 66, 269-279.
Subramaniam, A., Brown, C. W. Hood, R. R. Carpenter, E. J. and Capone, D. G. (2001). Detecting Trichodesmium blooms in SeaWiFS imagery. Deep-Sea Research II, 49(1-3), 107-121.
Taylor, R. (1997). That bloomin' Microcystis: Where'd it come from? Where'd it go? Twine Line, 19, 1.
Toledo Blade, (2002). Toxic Algae Thrive in Summer's Heat, Article ID: 0209170185, Published on Sep. 17, 2002, The Blade, Toledo, Ohio.
Vanderploeg, H. A., Liebig, J. R., Carmichael, W. W., Agy, M. A., Johengen, T. H., Fahnenstiel, G. L., Nalepa, T. F. (2001). Zebra mussel (Dreissena polymorpha) selective filtration promoted toxic Microcystis blooms in Saginaw Bay (Lake Huron) and Lake Erie. Canadian Journal of Fisheries and Aquatic Science, 58, 1208-1221.
Vincent, R. K. (1997). Fundamentals of Geological and Environmental Remote Sensing (pp. 102-108). Upper Saddle River, N.J.: Prentice Hall.
Vincent, R. K. (2000). Forecasts of monthly averaged daily temperature highs in Bowling Green, Ohio from monthly sea surface temperature anomalies in the Eastern Pacific ocean during the previous year. Photogrammetric Engineering & Remote Sensing, 66(8), 1001-1009.
Watras, C. J., Baker, A. L. (1988). Detection of planktonic cyanobacteria by tandem in vivo fluorometry. Hydrobiologia, 169, 77-84.
Welschmeyer, N. A. (1994). Fluorometric analysis of chlorophyll a in the presence of chlorophyll b and pheopigments. Limnology and Oceanography, 41, 1425-1437.

Having shown and described a preferred embodiment of the invention, those skilled in the art will realize that many variations and modifications may be made to affect the described invention and still be within the scope of the claimed invention. Thus, many of the elements indicated above may be altered or replaced by different elements which will provide the same result and fall within the spirit of the claimed invention. It is the intention, therefore, to limit the invention only as indicated by the scope of the claims.

What is claimed is:

1. A method of determining the presence of microcystin in water from light reflected therefrom, said method comprising the steps of:
(a) obtaining a measurement of reflected light from said water, said measurement comprising a measurement of the respective amount of light in at least four frequency ranges: (i) from about 0.45 μm to about 0.52 μm (ii) from about 0.63 μm to about 0.69 μm; (iii) from about 0.76 μm to about 0.90 m; and (iv) from about 1.55 μm to about 1.75 μm; and
(b) relating the approximate amount of said microcystin in said water to said respective amounts of light by applying an algorithm relating said respective amounts of light in said at least four frequency ranges to the amount of microcystin in said water.

2. A method according to claim 1 wherein said measurement of the amount of light in said at least four frequency ranges comprises the measurement, respectively, of: (i) LANDSAT TM band 1, (ii) LANDSAT TM band 3, (iii) LANDSAT TM band 4, and LANDSAT TM band 5.

3. A method according to claim 1 wherein the algorithm is any algorithm selected form the group consisting of X≈K₁+K₂×(R31)+K₃×(R43)+K₄×(R51), wherein X is the approximate amount of microcystin expressed in micrograms (μg) per liter (L) and wherein:
K₁ is a value in the range of from about −1 to about −89.7;
K₂ is a value in the range of from about 1 to about 186.99;
K₃ is a value in the range of from about 1 to about 132.99;
K₄ is a value in the range of from about −1 to about −179;
R31 is the value of LANDSAT TM band 3 divided by LANDSAT TM band 1, after subtraction for atmospheric haze separately in each band;
R43 is the value of LANDSAT TM band 4 divided by LANDSAT TM band 3, after subtraction for atmospheric haze separately in each band; and
R51 is the value of LANDSAT TM band 5 divided by LANDSAT TM band 1, after subtraction for atmospheric haze separately in each band.

4. A method according to claim 3 wherein:
K₁ is a value in the range of from about −25 to about −75;
K₂ is a value in the range of from about 50 to about 150;
K₃ is a value in the range of from about 45 to about 100;
K₄ is a value in the range of from about −50 to about −150;

5. A method according to claim 3 wherein:
K₁ is a value in the range of from about −50 to about −70;
K₂ is a value in the range of from about 85 to about 115;
K₃ is a value in the range of from about 55 to about 80; and
K₄ is a value in the range of from about −75 to about −100.

6. A method according to claim 3 wherein:
K₁ is a value of about −63.2388;
K₂ is a value of about 96.118;
K₃ is a value of about 68.362; and
K₄ is a is a value of about −92.006+.

7. A method according to claim 1 wherein the calculated value of microcystin correlates to the actual measured amount of said microcystin in said water by a correlation value in excess of 60%.

8. A method according to claim 1 wherein the calculated value of microcystin correlates to the actual measured amount of said microcystin in said water by a correlation value in excess of 70%.

9. A method according to claim 5 wherein the calculated value of X correlates to the actual measured amount of said microcystin in said water by a correlation value in excess of 60%.

10. A method according to claim 5 wherein the calculated value of X correlates to the actual measured amount of said microcystin in said water by a correlation value in excess of 70%.

11. A method according to claim 1 additionally comprising the step of transmitting data relating to the approximate amount of said microcystin in said water to a site remote from the site where said measurement takes place.

12. A method according to claim 5 additionally comprising the step of transmitting data relating to the approximate amount of said microcystin in said water to a site remote from the site where said measurement takes place.

13. A method according to claim 1 additionally comprising the step of generating a report of said approximate amount of said microcystin

14. A method of determining the presence of microcystin in water from light reflected therefrom, said method comprising the steps of:
(a) obtaining a measurement of reflected light from said water, said measurement comprising a measurement of the respective amount of light in at least four frequencies comprising, respectively: (i) LANDSAT TM band 1, (ii) LANDSAT TM band 3, (iii) LANDSAT band 4, and (iv) LANDSAT TM band 5; and (b) relating the approximate amount of said microcystin in said water to said respective amounts of light by applying an algorithm relating said respective amounts of light in said at least four frequency ranges to the amount of microcystin in said water, wherein said algorithm is any algorithm selected from the group consisting of X≈K₁+K₂×(R31)+K₃×(R43)+K₄×(R51), wherein X is the approximate amount of microcystin expressed in micrograms (μg) per liter (L) and wherein:
K₁ is a value in the range of from about −1 to about −89.7;
K₂ is a value in the range of from about 1 to about 186.99;
K₃ is a value in the range of from about 1 to about 132.99;
K₄ is a value in the range of from about −1 to about −179;
R31 is the value of LANDSAT TM band 3 divided by LANDSAT TM band 1, after subtraction for atmospheric haze separately in each band;
R43 is the value of LANDSAT TM band 4 divided by LANDSAT TM band 3, after subtraction for atmospheric haze separately in each band; and
R51 is the value of LANDSAT TM band 5 divided by LANDSAT TM band 1, after subtraction for atmospheric haze separately in each band.

15. A system for determining the presence of microcystin in water from light reflected therefrom, said device comprising:
(a) a measurement device adapted to measure reflected light from said water, said measurement comprising a measurement of the respective amount of light in at least four frequency ranges: (i) from about 0.45 μm to about 0.52 μm (ii) from about 0.63 μm to about 0.69 μm; (iii) from about 0.76 μm to about 0.90 m; and (iv) from about 1.55 μm to about 1.75 μm; and
(b) a processor capable of relating the approximate amount of said microcystin in said water to said respective amounts of light by applying an algorithm relating said respective amounts of light in said at least four frequency ranges to the amount of microcystin in said water.
